(19) United States
(12) Patent Application Publication
(10) Pub. No.: US 2005/0118271 A1
(43) Pub. Date: Jun. 2, 2005

(54) Polytartrate composition

(76) Inventors: Gesine Schliecker, Florsheim (DE); Carsten Schmidt, Mainz (DE); Stefan Fuchs, Essenheim (DE); Thomas Kissel, Staufen (DE)

Correspondence Address: AKZO NOBEL PHARMA PATENT DEPARTMENT, PO BOX 318, MILLSBORO, DE, 19966, US

(21) Appl. No.: 10/501,284

(22) PCT Filed: Jan. 15, 2003
(86) PCT No.: PCT/EP03/00514

(30) Foreign Application Priority Data
Jan. 16, 2002 (EP) 020751764

(51) Int. Cl. A61K 39/395; A61K 39/00; A61K 9/14

(52) U.S. Cl. 424/486; 424/130.1; 424/185.1; 514/12

(57) ABSTRACT

The invention provides a polytartrate composition for pulsatile release of a pharmaceutically active material that is in the form of a compressed tablet, and a process for preparing such a composition.

The present invention is concerned with a polytartrate composition for pulsatile release of a pharmaceutically active material, a process for preparing such a composition and the use thereof.

The modern medicinal therapy and prophylactics requires novel administration forms, which combine a controlled release rate of the pharmaceutically active material with high biocompatibility of the formulation. Different pharmaceutically active materials used in treatment of humans and animals require different release profiles. Pulsatile drug delivery is useful, for example, for the delivery of pharmaceutically active materials, that have short half-lives, and must be administered two or three times daily or with pharmaceutically active materials that are extensively metabolised pre-systemically or with pharmaceutically active materials, which loses the desired therapeutic effect when constant blood levels are maintained. It has long been appreciated that the release of certain pharmaceutically active materials in bursts or pulses at predetermined times following a single administration could have significant practical advantages in clinical or veterinary practice.

For example, an area of great interest for this type of delivery system is single-shot immunisation. In a classic immunisation regime, a single dose of a vaccine is delivered in one injectable or oral dose “primer” that is repeated one ore more times with “booster” doses for a long lasting immunity. Such multiple administration may not be practically feasible, especially for big numbers of livestock animals, e.g. chicken, pigs or cattle. A single-shot immunisation would deliver a second burst of antigen at a predetermined interval following a first burst, whereas the second burst would elicit a secondary immune response without the need for a second booster vaccination (repeat application).

Controlled delivery systems, which are capable of pulsatile release could be also useful for the delivery of hormones, especially for gonadotropins and growth hormones, because these hormones fail to produce their effects unless they are released intermittently. A potential use is in the field of livestock reproduction management, were e.g. follicle stimulating hormone (FSH) is currently applied to induce superovulation in cows.

A variety of compositions have already been developed to provide pulsatile release of various pharmaceutically active materials after oral and parenteral administration as described in e.g. the International, patent application WO 92/17165, WO 93/17662, WO 93/03159, WO 96/12466, in U.S. Pat. Nos. 5,260,069, No. 5,656,298 or 5,429,822. Materials, which have been proposed for such controlled release systems, are biodegradable polymers, particular polyesters that are derived from hydroxycarboxylic acids. Much prior art has been directed to polymers derived from alpha-hydroxycarboxylic acid, especially to lactic acid in both its racemic and optically active forms (PLA), to glycolic acid (PGA) and to copolymers (PLGA) thereof such as e.g. described in U.S. Pat. No. 3,773,919.

In particular, it is known that, for many pharmaceutically active materials such compositions for providing time controlled pulsatile release may be obtained by using a barrier technology that is placed around the active ingredient, that is designed to degrade or dissolve after a certain time interval. One approach is the encapsulation the pharmaceutically active material in a suitable 25 polymer, or by dispersing the pharmaceutically active materials in a matrix with one or more coatings to delay the release and determine the timing of the release.

Various complicated barrier structures are proposed, employing separate 30 coating steps or the use of membrane reservoir devices for a pulsatile release of the pharmaceutically active material from the device. These barrier systems require additional steps in the manufacturing process and therefore increase the costs of such a device and make the manufacturing process very complex. The manufacturing process should, however, preferably be simple, versatile and amenable to mechanisation and automatisation.

Another disadvantage of membrane reservoir compositions is the fact that the core active material can be released by dumping whenever the release-rate limiting membrane is ruptured. This could then result in the release of an undesired high, or even toxic, amount of the pharmaceutically active material. Another disadvantage is, that during the manufacturing process organic solvents are used that should be better avoided, especially in parenteral compositions, because of the risk of local irritation after administration caused by solvent residues.

It was therefore desirable to find a composition for pulsatile release of pharmaceutically active material that is easy, robust and cost effective to manufacture and does not require a complex barrier system.

In U.S. patent application Ser. No. 5,391,696 depot preparations of polycondensates, which contain tartaric acid derivatives are described, that showed a uniformly controllable active substance release with a strongly decreased “initial burst” when they are used for depot preparations of pharmaceuticals. Such depot preparations, such as e.g. microparticles, that are manufactured by spray drying, and rod-shaped implants, that are manufactured by extrusion are described. Such a depot preparation is a dosage form that provides a profile in which the drug is released over a prolonged interval, at a substantially steady rate of release per unit of time. Surprisingly the current inventors found that a polytartrate composition that is produced by simple compression releases the pharmaceutically active material in a pulsatile manner without the need of an additional barrier structure. Pulsatile release implies an initial first release followed by an almost release-free interval, after which a second dose of the pharmaceutically active material is released.

It was furthermore found, that such compositions, that overcome the drawbacks of prior art can be prepared using easy, robust and cost effective standard techniques that do not employ any solvents or heat and therefore do not lead to potential irritant solvent residues in the device or loss of activity of incorporated drugs such as peptides and hormones.

Therefore the present invention provides a pharmaceutical composition comprising a polytartrate polymer and at least one pharmaceutically active material characterised in that the composition is capable of releasing the pharmaceutically active material in a pulsatile manner, obtainable by forming a tablet with a compression force between 10 and 65 kN/cm².

The manufacturing conditions should be such that a surface of the dosage form or the polymer matrix is of such porosity, that the degradation products of the polytartrate polymer that get formed inside the composition due to polymer degradation diffuse through the surface at a lower rate than they are formed. In order to reach this the application a sufficient compression force is required.

In general, the process of compaction has several identifiable phases. When powders undergo compaction the first process to occur is a consolidation of the powders. During this consolidation phase the powder particles adopt a more efficient packing order. The second phase of the compaction process is elastic or reversible deformation. The third phase of compaction is plastic, or irreversible deformation of the powder bed. That is the compression with a reduction in volume. (Gennaro, Remington: The Science and Practice of Pharmacy” (20. Edition, 2000).

For manufacturing of a composition according to the invention a compression with significant reduction of the powder volume is necessary. Therefore the composition is formed at a compression force between 10 and 65 kN/cm_{2.}

Preferred is a compression force between 15 and 65 kN/cm², more preferred between 20 and 50 kN/cm², especially preferred between 25 and 50 kN/cm². The composition according to the present invention can be in general a solid composition in various forms that is suitable for the release in an aqueous environment. Solid means solid at 25° C.

In Gennaro, Remington: The Science and Practice of Pharmacy” (20. Edition, 2000) Chapter 45, tablets are defined as solid pharmaceutical dosage forms containing drug substances with or without suitable diluents that are prepared by either compression or compression moulding methods. Compressed tablets are manufactured by compression methods. These tablets are formed by compression and normally do not contain a special coating. They are made from powdered, crystalline, amorphous or granular materials, alone or optionally in combination with binders, disintegrants, controlled release polymers, lubricants, diluents and in many cases colorants.

The term “tablets” is used in herein encompasses solid pharmaceutical dosage forms for oral and parenteral administration to an animal or human body as well as for providing a topical formulation.

The polytartrates to be used in the current invention are biodegradable polycondensates, which contain monomers of tartaric acid derivatives, preferably branched tartaric acid derivatives. Such polytartrates are described in U.S. Pat. No. 5,391,696 incorporated herein by reference. The term tartaric acid (dihydroxysuccinic acid) as used in the present invention includes the two entantiomers (+)-tartaric and (−)-tartaric acid and the racemate and the optically inactive mesotartaric acid and mixtures thereof. Polytartrates with a molecular weight of at least 15000 g/mol may be useful in the current invention.

The degradation of the polytartrate polymer leads to the release of the pharmaceutically active material. An increasing concentration of degradation products inside the composition according to the invention can lead to an increase of the pressure inside the composition. Preferably the polytartrate polymer forms degradation products that increase the pressure inside the composition. Such degradation products can be liquid or gaseous. This can be considered to be one reason for the pulse release profile i.e. the self-bursting of the composition that is connected with the second “booster” release of the pharmaceutically active material.

Suitable polytartrate polymers are polycondensates, which contain at least one polytartrate that form during degradation water-soluble C1 to C10, preferably C1 to C4 degradation products that have a molecular weight below 100 Dalton. Such degradation products are preferably alcohol, aldehyde or ester or acetone, more preferred C1 to C4 alcohol, aldehyde or ester. Especially preferred are such compounds that form during degradation C1-C3 alcohol, such as methanol, ethanol, propanol or isopropanol or alternatively acetone.

Useful compounds are polycondensates which contain 2,3-O-alkylidenetartaric acid derivatives, 2,3-O-alkylidene-L-threitol, furo [2,5] groups or terephtalates. Such polycondensates are e.g. 2′,3′-(1′,4′-diethyl)-L-tartryl poly-(2,3-O-isopropylidene)-L-tartrate, 2′,3′-(1′,4′-diethyl)-L-tartryl polyfurandicarboxylate, poly-(2,3-(1,4-diethyl)-L-tartyl)terephthalate but not polyalkylenetartrate were the monomers of the polyester are tartaric acid with an alkylene diol OH—(CH₂)ₙ—OH wherein n=4, 8 or 10.

Preferred polytartrates are e.g. polycondensates of dimethyl tartrate, diethyl tartrate and diisopropyl tartrate or copolymers thereof. More preferred polytartrates are polycondensates of, on the one hand dimethyl tartrate, diethyltartrate, or diisopropyltartrate or copolymers thereof and on the other hand 2,3-O-alkylidene tartaric acid derivatives.

Especially preferred is 2′3′-(1′,4′-diethyl)-L-tartryl poly-(2,3-O-isopropylidene)-L-tartrate.

The benefit of biodegradable polymers is that a surgical removal of the device after parenteral administration is unnecessary. Biodegradable means that the components are degraded into toxicologically harmless components in the course of time under physiological conditions, which are either metabolised or excreted by the human or animal body. The predetermined time delay (delay time prior to second release of the pharmaceutically active material) is in general dependent upon the rate of degradation of the materials, the water absorption by the device and the dissolution of the degradation products.

A polymer can be amongst other features characterised by its glass transition temperature. The polytartrate polymer, to be used in the current invention has a glass transition temperature that is greater than 40° C., preferably between 40° C. and 60° C. The glass transition temperature (T_{g}) separates rubbery from glassy form behavior i.e. is that temperature at which an adhesive loses its flexibility and becomes hard, inflexible, brittle and “grasslike.” If flexibility is required the glass transition temperature can be lowered e.g. by means of plasticizers.

The pharmaceutically active material to be used in the current invention can be generally a recombinant pharmaceutical or veterinary agent that has prophylactic activity (i.e. preventing diseases or pathological symptoms) or that has an activity for treating or curing pathological symptoms/diseases in humans or animals (e.g. antiinflammatories). The pharmaceutically active material with prophylactic activity can be either a chemical (e.g. vitamins, minerals) or can be a biological e.g. antigen/antibody that e.g. triggers a protective immune response. The pharmaceutically active material to be used in the current invention is selected from one or more of antigens, antibodies or pharmaceutical substances. The pharmaceutically active material may comprise any native, synthetic or recombinant pharmaceutical or veterinary agent, or feed additive or supplement, including antigens, antibodies, antitoxins, nucleic acids, vaccines, cytokines, growth promoters, hormones, cancer cell inhibitory agents, immune stimulants or supressants, hypnotics, sedatives, tranquilisers, anti-asthmatics, antitussives, diuretics, anti-ulcer agents, anti-inflammatories, antiinfectives, anti-fungals, anti-viral agents, antiparasitics, vitamins, tonics, cardiovascular drugs, analgesics, stimulants, enzymes or minerals.

Preferably the pharmaceutically active material to be used in the current invention is a reproductive hormone, especially a gonadoliberin-GnRH releasing hormone (GnRH agonist) or an analogue. Such compounds can be e.g. used in the treatment of reduced fertility by ovarian dysfunction or for the induction of ovulation and improvement of conception rate in various animals e.g. cows, mares, ewes and rabbits. Examples of suitable gonadoliberis and its analogues are [D-Ser(Bu)⁶] gonadoliberin-(1-9) nonapeptide ethylamide (buserelin) or [D-Ser(Bu)⁶] AzaGly-gonadoliberin (azagly nafarelin).

The pharmaceutically active ingredient may comprise one type of pharmaceutically active material or may be a mixture of different pharmaceutically active materials. The process of the present invention is especially advantageous for the incorporation of heat sensitive pharmaceutically active material, as no heat stress is employed during the manufacturing process of the device according to the invention. By heat sensitive pharmaceutically active material such material is meant that loses its activity and/or degrades at temperatures above the glass transition temperature of the polytartrate polymer. Furthermore no organic solvents are employed during the manufacturing process that allows the use of pharmaceutically active material that is sensitive to organic solvents.

The composition of the present invention may also be combined with another dosage forms which will combine the release profile of the novel composition with that of the other dosage form.

The amount of pharmaceutically active material used in the composition will vary from subject to subject, depending on age, general condition of the animal or human, the severity of the condition being treated and the type of the pharmaceutically active material. In general, an effective amount of pharmaceutically active material is employed meaning a non-toxic but sufficient amount to provide the desired therapeutic effect. Thus, it is not possible to specify an exact “effective amount”. However, an appropriate “effective” amount in any individual case may be determined by a person skilled in the art using routine experimentation.

The composition according to the invention may optionally additionally comprise one or more of pharmaceutical acceptable excipients or adjuvants.

The term adjuvant is intended to include any substance, which is incorporated into or administered simultaneously with the immunogen, which potentiates the immune response in the subject. Adjuvants include but are not limited to mineral adjuvants e.g. aluminium hydroxide and aluminium phosphate or calcium phosphate, emulsions e.g. Freud's complete or incomplete adjuvant, microbial products e.g. BCG (attenuated Mycobacterium tuberculosis), lectins, saponins, immunostimulafing complexes or liposomes.

The pharmaceutical or veterinary excipients may be e.g. used to influence the hydrophilic or lipophilic properties of the composition. The composition according to the current invention may further comprise pharmaceutical excipients known in the art e.g. as described in “Gennaro, Remington: The Science and Practice of Pharmacy” (20. Edition, 2000), incorporated by reference herein. Such pharmaceutical excipients are e.g. binders (e.g. gum tragacanth, PVP, cornstarch), disintegrating agents (e.g. corn starch, potato starch), diluents (e.g. lactose) and/or lubricants (e.g. magnesium stearate).

All such components, carriers and excipients must be substantially pharmaceutically or veterinary pure and non-toxic in the amounts employed and must be biocompatible and compatible with the pharmaceutically active material. Biocompatible in the present specification means that all components of the composition should be physiologically tolerable and should not cause an adverse histological response.

For preparation of the pharmaceutical composition according to the invention an effective amount of the pharmaceutically active material is mixed with the polytartrate polymer.

This mixture is than shaped by compression e.g. by direct compression, or compression moulding e.g. in a single punch press to form tablets of the desired size and shape, that are capable of being administered to a human or animal. After compression, the tablets must have a number of additional attributes such as appearance, hardness, disintegration ability, appropriate dissolution characteristics and uniformity, which also are influenced both by the method of preparation and by the added tabletting excipients present in the composition.

The manufacturing process is performed at a temperature below the glass transition temperature of the polymer, preferably at room temperature and is characterised in that it includes the steps of:
a) mixing an effective amount of a pharmaceutically active material with the polytartrate polymer,
b) shaping the mixture by tabletting equipment to form compressed tablets by applying a compression force between 10 and 65 kN/cm².

A preferred method for forming the composition herein is by direct compression of a powdered mixture, alone or in combination with other excipients. Direct compression consists of compressing tablets directly from powdered material without modifying the physical nature of the material itself.

In more detail in a first step an effective amount of a pharmaceutically active material and the polytartrate polymer are mixed in a mixing equipment known in the art.

The mixture is than sieved, to separate oversized particles and agglomerates. In a second mixing step optionally additional tabletting excipients are added as e.g. a lubricant (magnesium stearate) and/or colloidal silica (Aerosil) for improving the flow characteristics in order to reach a suitable mixture for the compression step. Optionally a second sieving step separates oversized particles.

The mixture is than transferred to a tabletting equipment, e.g. a single punch press or a rotary tablet machine known in the art.

The tablet is formed by the pressure exerted on the mixture by the punches within the die by applying a compression force between 10 and 65 kN/cm².

The tablet assumes the size and shape of the punches and die used. The particular physical form of the tablets may vary according to the situation in which the system is used. The composition for administration directly to a human or animal body may be in any suitable shape including elongate, oval, round, capsule-form, square, triangular or cylindrical shape.

Preferably the composition is cylindrical in shape for implants, as to produce devices which are adapted for implantation using a conventional device.

The composition of the current invention may be placed in the body of an animal or human which is desired to treat by any suitable known in the art technique, for example parenterally by subcutaneous or intramuscular injection, or by surgical implantation using conventional clinical or veterinary techniques, by administration into body cavities, by transdermal route or orally.

Alternatively the composition may be placed in an aqueous environment of animals, e.g. in a fish or shrimp pond to release pharmaceutically active materials for administration to aquatic animals (e.g. bath application).

Pulsatile release delivery systems, were the drug is released in bursts separated by time intervals of low or no drug release is advantageous and desired in many veterinary and human applications e.g. for the administration of hormones and vaccines.

Copolymeric polytartate was found to be a suitable biodegradable pulsatile release agent.

In one preferred embodiment the composition according to the invention is implanted subcutaneously into a human or animal body. Implants are solid devices suitable for parenteral delivery and may be in a range of sizes for example from less than 1 mm diameter to several cm depending on the species.

Preferably the polytartrate are used in the form of an implant that was selected for easy and gentle manufacturing without application of heat or organic solvents.

The multiphase pulsatile release profile is characterized by an initial burst, releasing a first portion of the drug, a secondary “lag phase” of low or no release of the drug followed by a second burst, releasing a second portion of the drug.

The initial burst can be attributed to the immediate dissolution and release of drug entrapped in the implant surface. In consequence, the initial dose is released within a short period, preferably within 1-3 days.

The tablet size, especially the diameter has an effect on the initial burst. A reduced tablet diameter leads to an increase of the initial burst. This could be explained by the larger specific area of tablets with a smaller diameter. After the initial burst is finished, a secondary phase occurs in which no or only a small amount of the drug is released (“lag phase”). This can be explained by the chemical structure of the polytartrate: time is still needed for hydrolytic degradation of hydrophobic polymer side chains which allows finally absorption of water.

The “lag phase” is depending on various parameters e.g. the physicochemical properties of the drug, drug load, molecular weight of the polytartrate, copolymer ratio of the employed polytartrate and porosity and size of the tablet and can last e.g. between 9 and 11 days until the second burst occurs.

The second burst occurs in parallel with a dramatically change of the tablet shape. With increasing incubation time the original flat, circular tablet gets more and more bloated until bursting of the tablet.

In consequence, the booster dose is released within a short period, preferably 1-4 days. The observed bursting of the tablet occurs in parallel with accelerated mass loss.

The remaining polymer mass, which is no longer of regular size and shape, releases the remaining drug rather constant at a slow release rate.

In another embodiment, the composition according to the invention may also be administered orally. Where the composition of the current invention is to be administered by oral ingestion, particularly to ruminants, it may be incorporated into a weighed capsule or bolus or other intra-ruminal device.

The recipient of the composition may be a human, a livestock animal e.g. sheep, cattle, pig, goat, poultry, a laboratory test animal, e.g. a rabbit, guinea pig, rat or mouse or a companion animal e.g. dog, cat or horse, a fish, shrimp or another aquatic animals or a wild animal.

### EXAMPLE 1

### Preparation of (2′3′-(1′,4-diethyl)-L-tartryl poly-(2,3-o-isopropylidene)-L-tartrate (PTA) Buserelin-Tablets

TABLE 1

| Tablet | PTA | Buserelin-acetate |
|---|---|---|
| Buserelin 10% - PTA | 244.4 mg | 28.6 mg |
| tablet 5 mm | | |
| Buserelin 10% - PTA | 126.2 mg | 14.8 mg |
| tablet 3 mm | | |
| Buserelin 5% - PTA | 137.0 mg | 7.3 mg |
| tablet 3 mm | | |

Buserelin (INN) is a synthetic nonapeptide and an analogue to the hypothalamus hormone gonadotropin. The amounts of 2′3′-(1′,4′-diethyl)-L-tartryl poly-(2,3-o-isopropylidene)-L-tartrate (PTA) and of Buserelin-acetate as shown in Table 1 were triturated in an agate mortar to obtain a homogeneous mixture. Subsequently, the mixture was compressed in a single punch press using flat-faced punches of 3 mm at a compression force of 48 kN/cm₂ or 5 mm in diameter at a compression force of 27 kN/cm² The resulting tablets had a weight of 14 or 40 mg respectively. The size of the tablets has been determined with a calibrate vernier calipier.

### EXAMPLE 2

In vitro release of Buserelin from the PTA tablets Material and Methods: The tablets were prepared as described above. The weighted tablets were immersed in 12 ml phosphate buffer (0.05M, pH 7.4) containing 0.05% benzalconiumchloride and 0.1% sodium azide (release medium) and were incubated at 37° C. for 4 weeks. At defined time points 8 ml of the release medium were withdrawn and replaced by fresh medium. Samples were analysed for Buserelin content by HPLC, using reverse phase HPLC with UV detection at 220 nm.

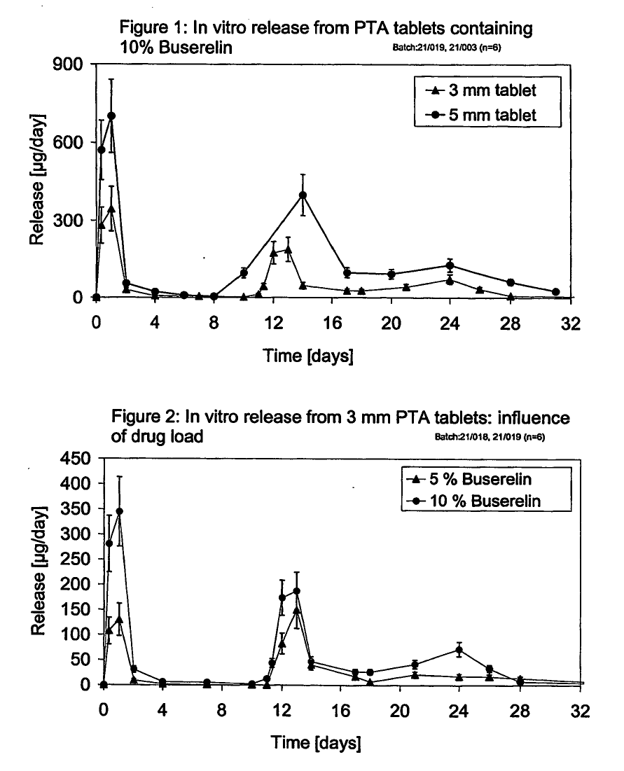

Results: FIG. 1 shows the release from PTA tablets of 3 mm and 5 mm diameter containing 10% Buserelin-acetate as a function of time. FIG. 2 shows the release from PTA tablets containing 10% and 5% Buserelin-acetate in a tablet of 3 mm diameter as a function of time.

When the tablets were incubated in the release medium at 37° C., after an initial burst, the release drops to very small amounts from day 3 to day 10. During the first 10 days neither a mass loss, nor water absorption were observed. After 10 days water absorption and mass loss commence. In parallel, a remarkable increase in drug release (“secondary burst”) occurs which continues over 2-4 days. The second burst occurred in parallel with a dramatically change of implant shape. With increasing incubation time the original flat, circular implant became more and more bloated until dehisce of the implant.

It was found that the extent of second burst “booster dose” is related to drug loading and increases with decreasing drug loading. This can be explained with the lower initial burst and the resulted higher drug content in implants containing low percentage of drug. A linear relationship between initial and second burst was found, which allows the adjustment of booster dose. The second burst was followed by a rather constant release up to the end of the release.

### EXAMPLE 3

### Release of Azagly-Nafarelin from the PTA Tablets in vivo in Pre-Dubertal Ewe-Lamb

Administration of GnRH agonists, such as buserelin or azagly-nafarelin, can trigger LH secretion which may induce terminal follicular growth and ovulation. LH secretion needs to be sustained for a few days to achieve this goal. The aim of this study was to assess safety and efficacy of polytartrate implants (PTA) releasing buserelin or azagly-nafarelin to induce terminal follicular growth and ovulation in an anovulatory model: the prepubertal ewe-lamb.

Material and Methods:

The polytartrate implants releasing buserelin or azagly-nafarelin were prepared according to the process described in Example 1. The tablets were compressed in a single punch press using flat-faced punches of 3 mm at a compression force of 46 kN/Cm².

| Tablet | PTA | Active ingredient |
|---|---|---|
| Azagly nafarelin - PTA | 140.0 mg | 1.0 mg |
| tablet 3 mm | | Azagly nafarelin acetate |
| Buserelin 10% - PTA | 126.2 mg | 14.8 mg |
| tablet 3 mm | | |

During the non-breeding season 15 prepubertal ewe lambs were randomly allocated to 2 treatment groups while one control group remained untreated.

To evaluate efficacy (induction of terminal follicular growth and ovulation) the azagly-nafarelin and LH profiles were characterized.

Treatment induced changes in LH concentrations of the PTA (polytartrate) treated groups, which were expected to release the GnRH analogue for a brief duration following implantation were monitored for only two days (day 0 and day 1). Treatment induced changes in azagly-nafarelin or buserelin concentrations were assessed in 6 h, 12 h, 15 h, 18 h and 24 h samples on day 0 and day 1 and then daily until day 16 for PTA and control groups.

The safety of each treatment was evaluated on the basis of rectal temperature and careful examination of the insertion site.

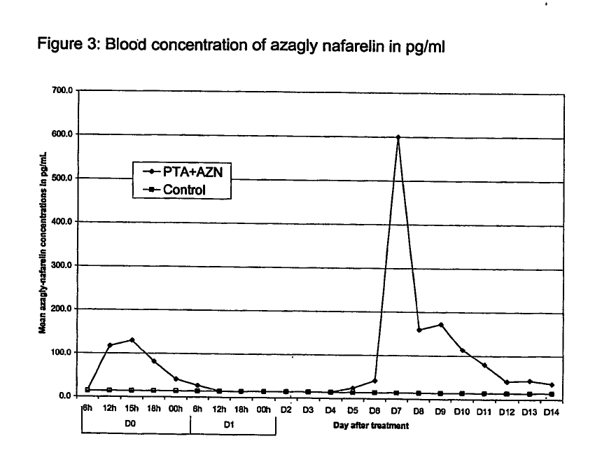

Results: Azagly-nafarelin release: The effects of time and treatment on azagly-nafarelin concentrations were evaluated. A highly significant time×treatment interaction (P<0.0001) was demonstrated. This was because a peak of azagly-nafarelin was observed 2 hours after treatment with PTA azagly-nafarelin implants, while azagly-nafarelin concentrations were low in the control group. A fairly synchronous second azagly-nafarelin peak was observed between days 7 and 9 in the PTA azagly-nafarelin group. Results are shown in FIG. 3.

LH concentrations remained low in the control group, while in the PTA azagly nafarelin and buserelin groups a LH peak was observed 2 hrs after treatment.

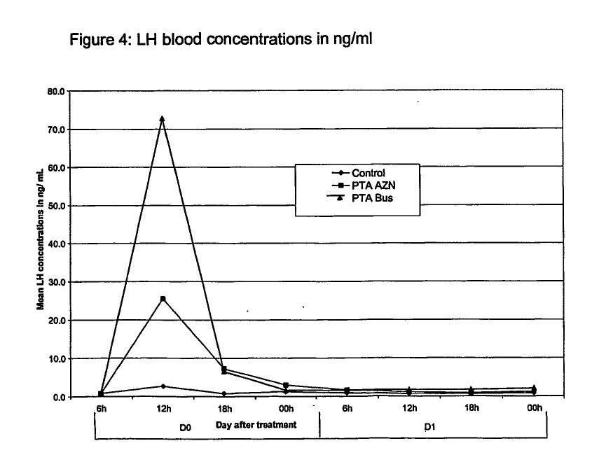

A significant time×treatment interaction (P=0.01) was detected as the LH peak was higher in PTA buserelin treated animals than in PTA azagly-nafarelin treated animals. Results are shown in FIG. 4.

Irrespective of treatment, no obvious change in rectal temperature was noticed. Irrespective of the group, no side effect was observed at the insertion site.

Conclusion: The PTA-Azagly nafarelin formulation presented In vivo an pulse release azagly-nafarelin profile with a first azagly-nafarelin peak a few hours after treatment followed by a delayed peak (around 7 days after treatment) and no azagly-nafarelin released in between these two peaks. Hence, the in-vivo results confirm the in-vitro results. Following treatment, all treated animals presented an immediate LH peak.

1. A pharmaceutical composition comprising a polytartrate polymer and at least one pharmaceutically active material characterised in that the composition is capable of releasing the pharmaceutically active material in a pulsatile manner and is obtainable by forming the tablet with a compression force between 10 and 65 kN/cm².

2. The composition according to claim 1 characterised in that the composition is formed at a compression force between 20 and 50 kN/cm².

3. The composition according to claim 1 characterised in that the polytartrate polymer forms degradation products that increase the pressure inside the composition.

4. The composition according to claim 3 characterised in that the polytartrate polymer forms during degradation a C1 to C4 alcohol, aldehyde or ester or acetone.

5. The composition according to claim 4 characterised that the polytartrate polymer forms during degradation methanol, ethanol, propanol, isopropanol or acetone.

6. The composition according to claim 1 characterised in that the polytartrate polymer is selected from the group of polycondensates of dimethyl tartrate, diethyl tartrate, diisopropyl tartrate or copolymers thereof and 2,3-O-alkylidenetartaric acid derivatives.

7. The composition according to claim 6 characterised in that the polytartrate polymer is 2′3′-(1′,4′-diethyl)-L-tartryl poly-(2,3-O-isopropylidene)-L-tartrate.

8. The composition according to the claim 1 characterised in that the polytartrate polymer has a glass transition temperature that is greater than 40° C.

9. The composition according to claim 1 characterised in that the pharmaceutically active material is selected from one or more of antigens, antibodies or pharmaceutical substances.

10. The composition according to claim 9 characterised in that the pharmaceutically active material is a GnRH agonist.

11. The composition according to claim 10 characterised in that the pharmaceutically active material is buserelin.

12. The composition according to claim 11 characterised in that the pharmaceutically active material is azagly nafarelin.

13. The composition according to claim 1 characterised in that the composition additionally comprises one or more of pharmaceutically acceptable excipients or adjuvants.

14. Process for the preparation of a polytartrate composition according to claim 1 involving the steps of
a) mixing an effective amount of a pharmaceutically active material with the polytartrate polymer,
b) shaping the mixture by a tabletting equipment to form compressed tablets by applying a compression force between 10 and 65 kN/cm².

15. according to claim 14 characterised in that the pharmaceutically active material and the polytartrate polymer are mixed in a powdered form.

16. The process according to claim 14 characterised in that the mixture is sieved and optionally additional tabletting excipients are added to the mixture.

17. A method of administering a pulsatile pharmaceutically active material to a body comprising the step of administering the composition of claim 1 to the body.

18. The method of claim 17 wherein the body is selected from an animal body and a human body.

19. A method of administering a pharmaceutically active material to a body comprising the steps of:
administering a composition of claim 1 to the body wherein the pharmaceutically is released in at least two phases comprising an initial burst and a second burst.

20. The method of claim 19 wherein the initial burst and the second burst is separated by a lag phase.
